(19) United States
(12) Patent Application Publication
(10) Pub. No.: US 2022/0304942 A1
(43) Pub. Date: Sep. 29, 2022

(54) TREATMENT OF OBESITY AND RELATED CONDITIONS

(71) Applicant: University of Greenwich, London (GB)

(72) Inventors: Elizabeth Shephard, London (GB); Sunil Veeravalli, London (GB); Jeremy Everett, London (GB); Dorsa Varshavi, London (GB)

(21) Appl. No.: 17/639,013

(22) PCT Filed: Sep. 1, 2020
(86) PCT No.: PCT/GB2020/052086

§ 371 Date: Feb. 28, 2022

(30) Foreign Application Priority Data
Aug. 30, 2019 (GB) 1912490.8

(51) Int. Cl. A61K 31/047; A61K 9/08; A61P 3/08; A61P 3/04

(52) U.S. Cl. CPC A61K 31/047; A61K 9/08; A61P 3/08; A61P 3/04; A61K 31/075

(57) ABSTRACT

The present invention relates to the use of specific compounds and compositions in the treatment or prevention of obesity, insulin resistance and diabetes and in the reduction of metabolic ageing. Also provided are related non-therapeutic uses for controlling the weight of an individual and for reducing metabolic ageing.

### FIELD OF THE INVENTION

The present invention relates to the treatment or prevention of obesity, insulin resistance and diabetes. In particular, it relates to the treatment or prevention of such conditions using the compound 2,3-butanediol and related compounds. The invention also relates to reduction of metabolic ageing.

### BACKGROUND TO THE INVENTION

Obesity is a medical condition in which excess body fat has accumulated to the extent that it may have a negative effect on health. Its worldwide prevalence is rapidly increasing, having more than doubled between 1980 and 2014, at which point approximately 600 million people globally were considered to be clinically obese.

Obesity is both a disease in its own right (for example, it is classified as such by the American Medical Association) and is associated with numerous comorbidities. For example, obesity increases the likelihood of various cardiovascular diseases, insulin resistance and diabetes, obstructive sleep apnea, certain types of cancer, osteoarthritis and depression. Insulin resistance (IR) is a pathological condition in which cells fail to respond normally to the hormone insulin, and is consequently associated with high blood glucose levels, and can contribute to the development of further pathological conditions such as type 2 diabetes. Diabetes is a group of metabolic disorders in which high blood sugar levels persist over a prolonged period and lead to numerous deleterious consequences. Metabolic age aligns the metabolic status of an individual to the average metabolic status of individuals having a given chronological age. A metabolic age exceeding chronological age may be associated with a range of undesirable effects on physical health, including but not limited to early death and an increased risk of conditions such as obesity, insulin resistance and diabetes (see, for example, Hertel et al., J. Proteome Research 15: 400-410 (2016).

At present, treatment options for obesity, including bariatric surgery and existing approved drugs, are limited and sub-optimal for a range of reasons, including concerns about efficacy and safety. There is an urgent need for further and improved treatments for obesity and related conditions, including insulin resistance and diabetes, to address the developing global public health crisis in this area.

Recent research described in Scott et al., Drug Metab Dispos 45:982-989 2017 has identified the gene Fmo5 as a therapeutic target for obesity and insulin resistance. However, no inhibitors of FMO5 have previously been identified or shown to have any effect on the metabolic processes associated with obesity, insulin resistance or diabetes.

### SUMMARY OF THE INVENTION

A metabonomic analysis has now identified a microbiome-derived compound, 2,3-butanediol, as being specifically present at high levels in the plasma and urine of FMO5 knock-out mice, with the corresponding wild type C57BL/6 mice generally having levels that are undetectable by NMR spectroscopy. Furthermore, treatment of wild-type mice with 2,3-butanediol ‘phenocopied’ several features of the FMO5 knock-out mice. For example, 2,3-butanediol in C57BL/6 mice caused significant reductions in fat to body-weight ratio, plasma cholesterol, triglycerides, free fatty acids and lactate dehydrogenase. These results indicate that 2,3-butanediol may be efficacious in the treatment of conditions such as obesity, insulin resistance and diabetes.

The present invention thus provides:
[1] 2,3-butanediol, or a physiologically acceptable ester, acetal or ketal thereof, for use as a medicament.
[2] 2,3-butanediol, or a physiologically acceptable ester, acetal or ketal thereof, for use in the treatment or prevention of obesity.
[3] 2,3-butanediol, or a physiologically acceptable ester, acetal or ketal thereof, for use in the treatment or prevention of insulin resistance.
[4] 2,3-butanediol, or a physiologically acceptable ester, acetal or ketal thereof, for use in the treatment or prevention of diabetes.
[5] 2,3-butanediol, or a physiologically acceptable ester, acetal or ketal thereof, for use of [4], wherein the diabetes is selected from the group consisting of type 2 diabetes, type 1 diabetes and gestational diabetes.
[6] 2,3-butanediol, or a physiologically acceptable ester, acetal or ketal thereof, for use of any one of [2] to [5], wherein the treatment or prevention is treatment.
[7] 2,3-butanediol, or a physiologically acceptable ester, acetal or ketal thereof, for use of any one of [2] to [5], wherein the treatment or prevention is prevention.
[8] 2,3-butanediol, or a physiologically acceptable ester, acetal or ketal thereof, for use in the therapeutic reduction of metabolic ageing.
[9] 2,3-butanediol, or a physiologically acceptable ester, acetal or ketal thereof, for use of any one of the preceding claims, wherein the 2,3-butanediol, or a physiologically acceptable ester, acetal or ketal thereof, is 2,3-butanediol.
[10] Non-therapeutic use of 2,3-butanediol, or a physiologically acceptable ester, acetal or ketal thereof, for controlling the weight of an individual.
[11] Non-therapeutic use of 2,3-butanediol, or a physiologically acceptable ester, acetal or ketal thereof, for reducing the metabolic ageing of an individual.
[12] A unit dose form suitable for oral administration to an individual, wherein the unit dose form contains 2,3-butanediol, or a physiologically acceptable ester, acetal or ketal thereof, in an amount of from 1 to 50 g, together with at least one physiologically acceptable excipient or carrier.
[13] The unit dose form of [12], wherein the unit dose form is formulated as a liquid.
[14] The unit dose form of [12] or [13], wherein the at least one physiologically acceptable excipient or carrier comprises at least one of a flavouring agent, a viscosity modifier, a surfactant, a preservative and a colouring agent.
[15] A method of treating or preventing a condition selected from the group consisting of obesity, insulin resistance and diabetes in an individual in need thereof, the method comprising administering to the individual a therapeutically effective amount of 2,3-butanediol, or a physiologically acceptable ester, acetal or ketal thereof.
[16] A method of controlling the weight of an individual, the method comprising administering to the individual 2,3-butanediol, or a physiologically acceptable ester, acetal or ketal thereof, in an amount sufficient to control said weight.
[17] A method of reducing the metabolic ageing of an individual, the method comprising administering to the individual 2,3-butanediol, or a physiologically acceptable ester, acetal or ketal thereof, in an amount sufficient to reduce said metabolic ageing.
[18] Use of 2,3-butanediol, or a physiologically acceptable ester, acetal or ketal thereof, in the manufacture of a medicament for use in the treatment or prevention of a condition selected from the group consisting of obesity, insulin resistance and diabetes, or for use in the therapeutic reduction of metabolic ageing.

Further aspects of the present invention are outlined in detail below.

### DETAILED DESCRIPTION

### 2,3-Butanediol

2,3-butanediol is a well-known organic compound having the structure

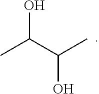

2,3-butanediol has been widely used in industry, for example in the preparation of polyurethanes. It is also known to be produced by various microorganisms in a process known as butanediol fermentation.

2,3-butanediol is found in various food products, such as cocoa butter, sweet corn, mussels and wine. For example, the levels of 2,3-butanediol in wine have been reported to range from about 0.2 to 3 g/L with a mean value of ca 0.6 g/L. These levels are sufficiently high to support the principle that 2,3-butanediol can safely be administered to humans in the quantities envisaged for the medical and non-medical (e.g. cosmetic) uses of the present invention.

2,3-butanediol has three stereoisomers, namely the [2S,3S] and [2R,3R] enantiomers and the [2S,3R] (or, equivalently, [2R,3S]) meso compound. In the present invention, the 2,3-butanediol can be present in any stereochemical form, inclusive of any single stereoisomer and any mixture of two or more of the three stereoisomers. For example, the 2,3-butanediol is optionally stereoisomerically pure or stereoisomerically enriched.

As used herein and unless otherwise indicated, the term “stereoisomerically pure” means a composition that comprises one stereoisomer of a compound and is substantially free of other stereoisomers of that compound. A typical stereoisomerically pure compound comprises greater than about 80% by weight of one stereoisomer of the compound and less than about 20% by weight of other stereoisomers of the compound, preferably greater than about 90% by weight of one stereoisomer of the compound and less than about 10% by weight of the other stereoisomers of the compound, more preferably greater than about 95% by weight of one stereoisomer of the compound and less than about 5% by weight of the other stereoisomers of the compound, and most preferably greater than about 98% by weight of one stereoisomer of the compound and less than about 2% by weight of the other stereoisomers of the compound.

As used herein and unless otherwise indicated, the term “stereoisomerically enriched” means a composition that comprises greater than about 55% by weight of one stereoisomer of a compound, preferably greater than about 60% by weight of one stereoisomer of a compound, more preferably greater than about 70% by weight, and most preferably greater than about 80% by weight of one stereoisomer of a compound.

As is well known in the pharmaceutical arts, physiologically (e.g. therapeutically) active compounds bearing one or more hydroxyl (—OH) groups, i.e. alcohols, can optionally be administered in form of a physiologically acceptable ester thereof. One particular well-known example of such a compound is aspirin, acetylsalicylic acid, which is the acetyl ester of its active metabolite, salicylic acid.

Thus, 2,3-butanediol can be provided in the form of a physiologically acceptable ester thereof. The physiologically acceptable ester is typically a compound capable of hydrolysing in vivo to yield the active alcohol as a metabolite, i.e. in this case 2,3-butanediol.

Suitable such esters would be readily apparent to those skilled in the art. Non-limiting examples include C₁₋₂₀ alkanoyl (preferably C₁₋₁₀ alkanoyl and more preferably C₂₋₆ alkanoyl) esters of 2,3-butanediol. Esterification may be at either or both (preferably both) of the hydroxyl groups of 2,3-butanediol.

For example, the 2,3-butanediol or a physiologically acceptable ester thereof may have the chemical formula

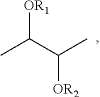

wherein R₁ and R₂ are the same or different and are each selected from H and —C(O)R₃ in which R₃ is C₁₋₂₀ alkyl (preferably C₁₋₁₀ alkyl and more preferably C₂₋₆ alkyl).

Furthermore, 2,3-butanediol can be provided in the form of a physiologically acceptable acetal or ketal thereof. Suitable such acetals and ketals would be readily apparent to those skilled in the art. Non-limiting examples include compounds of the chemical formula

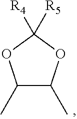

wherein R₄ and R₅ are the same or different and are each selected from H and C₁₋₂₀ alkyl (preferably C₁₋₁₀ alkyl and more preferably C₂₋₆ alkyl).

The physiologically acceptable acetal or ketal is typically a compound capable of hydrolysing in vivo to yield 2,3-butanediol.

Additionally, the 2,3-butanediol can be provided in the form of a physiologically acceptable salt or solvate thereof, or a prodrug thereof. References herein to 2,3-butanediol therefore generally embrace a salt or solvate thereof and a prodrug thereof (including in the claims, but not in the working examples or where sense otherwise dictates the contrary).

As used herein, “salts” include derivatives of an active agent, wherein the active agent is modified by making acid or base addition salts thereof. Such salts include, but are not limited to, physiologically acceptable acid addition salts, physiologically acceptable base addition salts, physiologically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, and ketoglutarates. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, and basic amino acids, e. g., lysine and arginine dicyclohexylamine. Examples of metal salts include lithium, sodium, potassium and magnesium salts. Examples of ammonium and alkylated ammonium salts include ammonium, methylammonium, dimethylammonium, trimethylammonium, ethylammonium, hydroxyethylammonium, diethylammonium, butylammonium and tetramethylammonium salts. Examples of organic bases include lysine, arginine, guanidine, diethanolamine and choline. Standard methods for the preparation of physiologically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa. (2003).

As used herein, “solvate” means a complex formed by solvation (the combination of solvent molecules with molecules or ions of the active agent of the present invention), or an aggregate that consists of a solute ion or molecule (the active agent of the present invention) with one or more solvent molecules. A preferred solvate is a hydrate. Examples of hydrate include, but are not limited to, hemihydrate, monohydrate, dihydrate, trihydrate, hexahydrate, etc. It should be understood by one of ordinary skill in the art that the physiologically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometrically related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal lattice. Preferred are physiologically acceptable solvents.

As used here, “prodrug” means a derivative of an active agent (in the present case, a derivative of 2,3-butanediol) that is suitable for administration to a subject and which is capable of being metabolised in vivo following administration to generate the active agent (in the present case, 2,3-butanediol).

### Individual to be Treated

Typically the individual to be treated is a mammal. Preferably the individual is a human. However, the individual may also be a non-human animal, for example a non-human mammal such as, but not limited to, a dog, a cat or a horse.

A human individual may be either an adult (e.g. aged 19 years or above) or a child (e.g. of age of less than 19 years).

### Conditions to be Treated or Prevented

One aspect of the present invention relates to the treatment or prevention of obesity. Obesity is a pathological condition. It is a disease, as confirmed for example by the fact it is classified as such by the American Medical Association. The obesity is typically clinical obesity. The obesity to be treated or prevented can be any obesity identified as such by a medical professional.

As is well known in the art, body mass index (BMI) is one commonly used screening tool for diagnosing whether an individual suffers from obesity. BMI is defined as an (human) individual's weight in kilograms divided by the square of his or her height in metres giving a BMI value measured in kg/m². In the present invention, obesity can, for example, be defined in accordance with WHO criteria, i.e. in an adult as an individual with a BMI greater than or equal to thirty. Alternatively or additionally, the adult may have a waist circumference of greater than or equal to 80 cm (for a female adult individual) or greater than or equal to 94 cm (for a male adult individual).

For a child, obesity is preferably diagnosed by a medical professional owing, for example, to the difficulty of developing a simple index for the measurement of obesity because of the number of physiological changes being undergone by children's bodies as they grow. In one embodiment, however, a child suffering from obesity is a child having a BMI above the 95^{th} percentile of a child of the same age and sex (reference here, and throughout, to the BMI of “a child of the same age and sex” means the mean BMI of children of that age and sex, e.g. in the country of birth of the child-to-be-treated).

In the present invention, the treatment or prevention of obesity preferably involves treatment of obesity, i.e. administration of the active agent commences on an individual who (already) suffers from obesity. However, the present invention also encompasses prevention (otherwise known as preventative treatment). For example, administration of the active agent may commence in a patient who does not suffer from obesity, but is considered (e.g. by a medical professional) to be on a trajectory to develop obesity, or at risk of developing obesity, unless preventative action is taken. Under such circumstances the individual may, for example, be overweight, but not obese. For example, an overweight but not obese adult may be one having a BMI of 25 to less than 30 (this corresponding to the WHO definition of “overweight”); an overweight, but not obese child may be one having a BMI from the 85^{th} percentile to less than the 95^{th} percentile of a child of the same age and sex e.g. in the country of birth of the child-to-be-treated.

Another aspect of the invention relates to treatment or prevention, preferably treatment, of insulin resistance (IR). Insulin resistance (IR) is a pathological condition in which cells fail to respond normally to the hormone insulin. When the body produces insulin under conditions of insulin resistance, the cells are resistant to the insulin and are unable to use it as effectively, leading to high blood sugar levels. Beta cells in the pancreas subsequently increase their production of insulin, further contributing to a high blood insulin level. Insulin resistance is known to contribute to the development of type 2 diabetes or latent autoimmune diabetes. Individuals who develop type 2 diabetes usually pass through earlier stages of insulin resistance.

The development of insulin resistance is commonly associated with, inter alia, the following factors: an individual being overweight or obese; an individual having a high-calorie diet, high-carbohydrate or high-sugar diet; an individual having a sedentary lifestyle; an individual taking steroids over a chronic period; an individual suffering from chronic stress; an individual having Cushing's disease or polycystic ovary disease. The treatment or prevention of insulin resistance may comprise treatment or prevention of insulin resistance in an individual having one or more of these characteristics.

Insulin resistance can routinely be diagnosed by a medical professional. Insulin resistance may, for example, be diagnosed by any of the sets of criteria described in the Medscape Insulin Resistance Guidelines, by Olatunbosun et al., updated Sep. 18, 2017 and available at https://emedicine.medscape.com/article/122501-guidelines.

A still further aspect of the present invention relates to the treatment or prevention of diabetes (otherwise known as Diabetes mellitus or simply “DM”). All forms of diabetes may be susceptible to treatment or prevention in accordance with the present invention. For example, the diabetes may be selected from type 2 diabetes, type 1 diabetes and gestational diabetes. Preferably, the diabetes is type 2 diabetes.

Increasingly it is recognised that there is a spectrum of types of diabetes in human patients and these may not be categorised neatly as type 1, type 2 or gestational diabetes. A yet further aspect of the present invention relates to the treatment or prevention of a spectrum of diabetic conditions that do not necessarily fall into the conventional descriptions of the disease.

A still further aspect of the invention relates to therapeutic reduction of metabolic ageing, i.e. a method of medical treatment comprising reducing metabolic ageing.

Ageing in general is an inevitable process in life; it is associated with the increased prevalence of a wide range of pathological conditions including, but not limited, to metabolic, inflammatory, cardiovascular and neurodegenerative diseases. Age in a chronological sense (i.e., as a simple measurement of time from birth) is thus a primary risk factor in a wide range of clinical conditions. Ageing is also well-established as being associated with a gradual decline in metabolic function.

Metabolic age is a somewhat different characteristic from the chronological age of a living subject. Metabolic age in particular may relate to the status of one or more biomarkers that correlate, on average, to a chronological age in a given population of living subjects (Hertel et al, J. Proteome Research 15: 400-410 (2016). For example, for a given biomarker or group of biomarkers A that is known to correlate with chronological age, then, for a population of subjects, each sub-set of subjects of a single chronological age in years will have a single average (e.g. typically mean average) status of A. Within each sub-set of patients of a specific chronological age, however, certain patients will typically have a status of biomarker or biomarker group A that corresponds to the average status of A of subjects of a lower chronological age (i.e. they will have a lower metabolic age than chronological age). Similarly, within each sub-set of patients of a specific chronological age, certain patients will typically have a status of A that corresponds to the average status of A of subjects of a higher chronological age (i.e. they will have a higher metabolic age than chronological age). Having a high metabolic age (particularly, but not limited to, one that exceeds chronological age) may be physiologically undesirable as it may be associated with a higher risk of onset of disease states associated with ageing. Furthermore, reducing metabolic aging is therapeutically desirable, since it may be associated with a decreased risk of the onset of pathological conditions associated with ageing.

Typically as a subject ages in chronological terms then his or her metabolic age also increases. However, metabolic ageing may occur faster, slower, or at the same rate as chronological ageing. Administration of the compounds identified herein to a subject can reduce metabolic ageing (for example, can achieve a lower metabolic age after a period of administration than would be the metabolic age in the absence of administration). Thus, by “reduction of metabolic aging” is meant that the subject's metabolic age is reduced relative to the expected metabolic age for a subject starting with the same metabolic age but undergoing no administration of the active agent.

Quantification of the metabolic age of a subject and monitoring of changes in metabolic age over time (i.e. as a subject ages chronologically) can, in general, be performed by reference to any biomarker or group of biomarkers (weighted in any amount) that are known to be correlated with metabolic age of the species (e.g. humans) to which the subject belongs (Hertel et al, J. Proteome Research 15: 400-410 (2016). As would readily be appreciated, the biomarker or group of biomarkers may be pre-calibrated to align a given status thereof (e.g. parameterised by a numerical value) against a particular chronological age (e.g., in years) of a population of subjects having the same status. In other words, a particular status of the biomarker or group thereof is associated with a particular chronological age in the population. In carrying out the present invention, an initial assessment can thus be made of the metabolic age of the subject to-be-treated (by measuring the status of the biomarker or group thereof in the specific patient and matching it to the age of subjects in the pre-calibration population having the same status). The rate of change of metabolic age can then be assessed by monitoring the rate of change of the same biomarker or group thereof in the patient over time. Reduction of metabolic ageing can, for example, correspond to a slowdown in the metabolic ageing in the subject, as compared to the metabolic ageing that would be anticipated based on the pre-calibrated population as a whole.

Examples of biomarkers that may be used to quantify metabolic age include glycolate, histidine, ethanolamine and threonine, which were found to be the main metabolites contributing to the metabolic age score in the Study of Health in Pomerania (SHIP-0) cohort of 4,308 Caucasian subjects residing in northeastern Germany (Hertel et al, J. Proteome Research 15: 400-410 (2016), the content of which is herein incorporated by reference in its entirety). However, more complex arrays of metabolic biomarkers can be used. Indeed, in the same study of the SHIP-0 cohort, 59 metabolites were used to create gender-specific metabolic age scores. In one non-limiting but exemplary embodiment of the present invention, reduction in metabolic age in a subject of a particular gender is measured with reference to the corresponding gender-specific metabolic age score described in this study (see, in particular, the equations S-1 on pages S13-S16 of the Supporting Information section of Hertel et al, J. Proteome Research 15: 400-410 (2016)).

Thus, in a typical study to investigate the therapeutic benefit of a treatment with 2,3-butanediol (or a physiologically acceptable ester, acetal or ketal thereof) on metabolic ageing, a cohort of suitably-selected and phenotyped individuals would be randomly assigned between treatment and control groups. Care would be taken to ensure that the two groups were as matched as closely as possible in terms of individual's chronological age, gender, BMI, waist circumference, smoking habit, drug intake, diet and drinking habits. The metabolic age score for each individual in each group would be quantified using a metabolic age score as described in Hertel et al, J. Proteome Research 15: 400-410 (2016) at the beginning of the study. The treatment group would then be administered 2,3-butanediol (or a physiologically acceptable ester, acetal or ketal thereof) at an efficacious dose for a suitable period which could be weeks to months or more, whilst the control group would be administered an identical-appearing placebo treatment over the same period of time. At the end of the study, the metabolic age score of each individual would be measured again and a two-tailed t-test would be used as a simple measurement of whether or not there has been a significant reduction in metabolic age increase relative to chronological age increase in the treated versus the placebo group. The expectation would be that the increase in metabolic age would equal the increase in chronological age in the placebo-treated control group. However, in the group treated with 2,3-butanediol (or a physiologically acceptable ester, acetal or ketal thereof), the expectation would be that the increase in metabolic age is (significantly) less than the increase in chronological age over the course of the experiment.

### Non-Therapeutic Use and Method

The 2,3-butanediol, or a physiologically acceptable ester, acetal or ketal thereof, can also be used in situations that are not directly, or are not, therapeutic in nature (e.g. cosmetically). For example, one aspect of the present invention involves non-therapeutic (e.g. cosmetic) use of 2,3-butanediol, or a physiologically acceptable ester, acetal or ketal thereof, for controlling the weight of an individual. The invention also provides a method (e.g. a cosmetic and/or non-therapeutic method) of controlling the weight of an individual, the method comprising administering to the individual 2,3-butanediol, or a physiologically acceptable ester, acetal or ketal thereof, in an amount sufficient to control said weight.

The individual undergoing such administration of the active agent in the use or method typically does not suffer from obesity. The use and method are therefore typically not a therapeutic or medical use or method; the use and method are non-therapeutic. The use and method typically do not involve prevention or treatment of (clinical) obesity.

The individual undergoing such administration of the active agent is typically not underweight. As with the diagnosis of obesity, the diagnosis of a patient as underweight may be performed by a medical professional or may be assessed in accordance with established classifications, such as body mass index. For example, an underweight adult may be defined as an adult having a BMI of less than 18.5. An underweight child may be defined as a child having a BMI less than the 5^{th} percentile of a child of the same age and sex.

The individual undergoing such administration of the active agent may be in the “normal or healthy” weight range or may be “overweight, but not obese”. Preferably such an individual is overweight, but not obese. For example, for an adult the individual may have a BMI of 18.5 to less than 30 (encompassing each of normal and overweight), or more preferably a BMI of 25 to less than 30 (overweight). For a child the individual may have a BMI from the 5^{th} percentile to less than the 95^{th} percentile of a child of the same age and sex (encompassing each of normal and overweight), or more preferably a BMI from the 85^{th} percentile to less than the 95^{th} percentile of a child of the same age and sex (overweight).

The individual undergoing such administration is preferably at least 16 years old, for example at least 18 years old or at least 19 years old.

Another aspect of the present invention involves non-therapeutic (e.g. cosmetic) use of 2,3-butanediol, or a physiologically acceptable ester, acetal or ketal thereof, for reducing the metabolic ageing of an individual. The invention also provides a method (e.g. a cosmetic and/or non-therapeutic method) of reducing the metabolic ageing of an individual, the method comprising administering to the individual 2,3-butanediol, or a physiologically acceptable ester, acetal or ketal thereof, in an amount sufficient to reduce said metabolic ageing. Non-therapeutic reduction of metabolic ageing may be desirable, for example, for aesthetic reasons or for other reasons not associated with direct therapeutic benefit. Preferably such non-therapeutic aspects again involve subjects who are at least 16 years old, for example at least 18 years old or at least 19 years old. Characterisation of the reduction of metabolic ageing can, in general, be undertaken in the same way as when the reduction of metabolic ageing is undertaken therapeutically (thus, the discussion elsewhere herein regarding characterising metabolic ageing applies also to these non-therapeutic aspects).

These non-therapeutic uses and methods are, in one preferred embodiment, nutraceutical uses and nutraceutical methods, respectively. Nutraceutical uses and nutraceutical methods may involve the administration of the 2,3-butanediol, or a physiologically acceptable ester, acetal or ketal thereof, in a form of a pharmaceutical-grade product. For example, such a product may be formulated as a dietary supplement, a medical food and/or as a functional food. A dietary supplement is a product taken by mouth that contains an ingredient intended to supplement the diet (in this case, the ingredient comprising or consisting of 2,3-butanediol, or a physiologically acceptable ester, acetal or ketal thereof). A functional food is a food item that additionally comprises an ingredient intended to provide a physiological benefit (again, the ingredient in this case comprising or consisting of 2,3-butanediol, or a physiologically acceptable ester, acetal or ketal thereof).

### Unit Dose and Pharmaceutical and Non-Pharmaceutical Compositions

The 2,3-butanediol, or a physiologically acceptable ester, acetal or ketal thereof, may be administered to an individual in a unit dose. The unit dose may, for example, be a unit dose suitable for use as a daily dose (i.e. the unit dose may contain the 2,3-butanediol, or a physiologically acceptable ester, acetal or ketal thereof, in an amount sufficient to deliver a therapeutically effective amount thereof when administered as the sole dose thereof in any given, single day). A dosage regimen may, of course, involve administering one such unit dose suitable for use as a daily dose per day for a plurality of days, as discussed in further detail elsewhere herein.

A unit dose typically contains 1 to 50 g of 2,3-butanediol, or a physiologically acceptable ester, acetal or ketal thereof. The 2,3-butanediol, or a physiologically acceptable ester, acetal or ketal thereof, is typically of pharmaceutical grade (i.e. it does not contain a significant level of impurities, particularly impurities that may not be physiologically acceptable). More preferably the unit dose contains 5 to 30 g and more preferably still 10 to 25 g of 2,3-butanediol, or a physiologically acceptable ester, acetal or ketal thereof.

The unit dose also contains at least one physiologically acceptable excipient or carrier. For the avoidance of doubt, such a physiologically acceptable excipient or carrier may comprise or consist of water. The physiologically acceptable excipient or carrier may comprise or consist of a flavouring agent, e.g. a sweetener. The physiologically acceptable excipient or carrier may comprise or consist of a preservative (e.g. an anti-microbial preservative). The physiologically acceptable excipient or carrier may comprise or consist of a colouring agent. The physiologically acceptable excipient or carrier may comprise or consist of a viscosity modifier. The physiologically acceptable excipient or carrier may comprise or consist of a surfactant (particularly preferably when the pharmaceutical composition is an emulsion). In one embodiment, the at least one physiologically acceptable excipient or carrier comprises at least two of a flavouring agent, a viscosity modifier, a preservative, a surfactant and a colouring agent (e.g. at least three of these components, at least four of these components, or up to all five of these components). Such a physiologically acceptable excipient or carrier may additionally comprise water.

The unit dose may be formulated in any physical form, but is typically either a liquid or a solid. Preferably the unit dose is formulated as a liquid.

The unit dose form may be contained in a sterile container.

For the avoidance of doubt, the unit dose identified herein is suitable for use in both the therapeutic and non-therapeutic aspects of the present invention. Thus, in one embodiment the unit dose is for use, or is suitable for use, (i) as a medicament, (ii) in the treatment or prevention of obesity, (iii) in the treatment or prevention of insulin resistance, (iv) use in the treatment or prevention of diabetes, or (v) in the therapeutic reduction of metabolic ageing. In another embodiment, the unit dose is for, or is suitable for, (i) non-therapeutic use for controlling the weight of an individual, or (ii) reducing the metabolic ageing of an individual. The unit dose may, for example, therefore be a nutraceutical product (e.g. a dietary supplement, medical food and/or a functional food).

The present invention still further extends to a pharmaceutical composition that has features analogous to those of the unit dose defined here, but without the limitation in the amount of active agent present. Such a pharmaceutical composition contains 2,3-butanediol, or a physiologically acceptable ester, acetal or ketal thereof, and also contains at least one physiologically acceptable excipient or carrier. Such a physiologically acceptable excipient or carrier may comprise or consist of water. The physiologically acceptable excipient or carrier may comprise or consist of a flavouring agent, e.g. a sweetener. The physiologically acceptable excipient or carrier may comprise or consist of a preservative (e.g. an anti-microbial preservative). The physiologically acceptable excipient or carrier may comprise or consist of a colouring agent. The physiologically acceptable excipient or carrier may comprise or consist of a viscosity modifier. The physiologically acceptable excipient or carrier may comprise or consist of a surfactant (particularly preferably when the pharmaceutical composition is an emulsion). In one embodiment, the at least one physiologically acceptable excipient or carrier comprises at least two of a flavouring agent, a viscosity modifier, a preservative, a surfactant and a colouring agent (e.g. at least three of these components, at least four of these components, or up to all five of these components). Such a physiologically acceptable excipient or carrier may additionally comprise water. The pharmaceutical composition may be formulated in any physical form, but is typically either a liquid or a solid. Preferably the pharmaceutical composition is formulated as a liquid.

The pharmaceutical composition is typically for use in the therapeutic aspects of the present invention. Thus, in one embodiment the pharmaceutical composition is for use, or is suitable for use, (i) as a medicament, (ii) in the treatment or prevention of obesity, (iii) in the treatment or prevention of insulin resistance, (iv) use in the treatment or prevention of diabetes, or (v) in the therapeutic reduction of metabolic ageing.

The present invention still further provides a composition that has features analogous to those of the pharmaceutical composition defined herein, but which is not earmarked for therapeutic use. Such a composition contains 2,3-butanediol, or a physiologically acceptable ester, acetal or ketal thereof, and also contains at least one physiologically acceptable excipient or carrier. It is interchangeably referred to herein as a “non-pharmaceutical composition” or “nutraceutical composition”. Such a non-pharmaceutical composition can be used, for example, for, (i) non-therapeutic use for controlling the weight of an individual, or (ii) reducing the metabolic ageing of an individual. For the avoidance of doubt, the non-pharmaceutical composition has the same physical properties (e.g. relating to constituents, and physical forms) as those discussed above in relation to the pharmaceutical composition.

### Administration

In general, the 2,3-butanediol, or a physiologically acceptable ester, acetal or ketal thereof, is administered in the form of a pharmaceutical or non-pharmaceutical composition (depending on the purpose of the administration; herein referred to collectively simply as a “composition”). The composition may in its simplest form comprise pure 2,3-butanediol, or a physiologically acceptable ester, acetal or ketal thereof (typically to pharmaceutical grade). The composition may alternatively additionally comprise at least one physiologically acceptable excipient or carrier. Compositions comprising the 2,3-butanediol, or a physiologically acceptable ester, acetal or ketal thereof, include, but are by no means limited to, the unit doses described elsewhere herein.

In general compositions used in the invention may be suitable for administration by any mode known in the art, e.g. oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal, or transcutaneous administration.

For oral administration, the compositions of the present invention may take the form of, for example, tablets, lozenges or capsules prepared by conventional means with physiologically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycolate); or wetting agents (e.g. sodium lauryl sulphate). The tablets may be coated by methods well known in the art. Liquid preparations for oral administration may take the form of, for example, solutions, syrups, emulsions or suspensions, or they may be presented as a dry product for constitution with water or other suitable vehicle before use. Where the active agent is itself a liquid under ambient conditions (e.g. 2,3-butanediol itself) then the liquid preparation may consist of pure 2,3-butanediol (or a physiologically acceptable ester, acetal or ketal thereof) or 2,3-butanediol (or a physiologically acceptable ester, acetal or ketal thereof) mixed with other liquids and/or 2,3-butanediol (or a physiologically acceptable ester, acetal or ketal thereof) in which other substances are dissolved and/or suspended.

Such liquid preparations may be prepared by conventional means with physiologically acceptable additives such as suspending agents, emulsifying agents, non-aqueous vehicles or preservatives. The preparations may also contain buffer salts, flavouring agents or colouring agents, as appropriate.

For ophthalmic administration the compositions of the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compositions of the present invention may be conveniently formulated as suppositories. These can be prepared by mixing the active component with a suitable non-irritating excipient that is solid at room temperature but liquid at rectal temperature and so will melt in the rectum to release the active component. Such materials include, for example, cocoa butter, beeswax and polyethylene glycols.

For topical administration the compositions of the present invention may take the form of any formulation normally used for topical administration, in particular solutions, lotions, emulsions of liquid consistency, emulsions of semi-liquid consistency, emulsions of semi-solid consistency, emulsions of solid consistency, creams, gels or ointments. The emulsions are obtained by dispersion of an oil phase in water (O/W) or a water phase in oil (W/O). For example, some compositions for topical administration contain an oil phase. Such compositions may, for example, be water-in-oil emulsions (i.e. emulsions wherein the water is the dispersed phase and the oil in the dispersion medium) or be substantially non-aqueous.

Compositions for topical use in accordance with the invention may also contain one or more emollients, emulsifiers, thickeners and/or preservatives. The emollients are typically long chain alcohols, such as cetyl alcohol, stearyl alcohol and cetearyl alcohol; hydrocarbons such as petrolatum and light mineral oil; or acetylated lanolin. The total amount of emollient in the formulation is preferably about 5% to about 30%, and more preferably about 5% to about 10% by weight based on the total weight of the formulation. The emulsifier is typically a nonionic surface active agent, e.g., polysorbate 60 (available from Sigma Aldrich), sorbitan monostearate, polyglyceryl-4 oleate, and polyoxyethylene(4)lauryl ether or trivalent cationic. Generally the total amount of emulsifier is preferably about 2% to about 14%, and more preferably about 2% to about 6% by weight based on the total weight of the formulation. Physiologically acceptable thickeners, such as Veegum™ K (available from R. T. Vanderbilt Company, Inc.), and long chain alcohols (i.e. cetyl alcohol, stearyl alcohol or cetearyl alcohol) can be used. The total amount of thickener present is preferably about 3% to about 12% by weight based on the total weight of the formulation. Preservatives such as methylparaben, propylparaben and benzyl alcohol can be present in the formulation.

Optionally, an additional solubilizing agent such as benzyl alcohol, lactic acid, acetic acid, stearic acid or hydrochloric acid can be included in the formulation. If an additional solubilizing agent is used, the amount present is preferably about 1% to about 12% by weight based on the total weight of the cream.

Optionally, the formulation can contain a humectant such as glycerin and skin penetration enhancers such as butyl stearate.

It is known to those skilled in the art that a single ingredient can perform more than one function in a composition, i.e., cetyl alcohol can serve both as an emollient and as a thickener.

The composition of the invention optionally comprises an oil phase. In this case, typically the amount of oil in the composition is at least 10 wt. %, preferably at least 30 wt. %, more preferably at least 50 wt. %, more preferably at least 80 wt. %, based on the total weight of the composition. As used herein an oil phase is typically a liquid or solid phase which is substantially immiscible with water. More typically, an oil phase as used herein has a solubility in water at 25° C. of less than or equal to 1 mg/L, preferably less than 0.1 mg/L.

The oil phase in an emulsion may be any oil phase normally used in emulsions for topical administration. Such oil phases include, for example, hydrocarbon bases such as such as hard paraffin, soft paraffin, ceresine and microcrystalline wax, absorption bases such as lanolin and beeswax, emulsifying bases such as emulsifying wax and cetrimide, and vegetable oils such as olive oil, coconut oil, sesame oil, almond oil and peanut oil. Other oil phases useful in accordance with the invention are mineral oil, liquid petroleum, sorbitan monostearate, polysorbate 60, cetyl esters wax, cetearyl alcohol, benzyl alcohol and 2 octyldodecanol.

Those skilled in the art will understand that by varying the ratio of water to oil in an emulsion, the result could be deemed a lotion, a cream, or an ointment, by order of increasing proportion and nature of the oil. An emulsion comprising similar proportions of oil phase and water phase is usually deemed a cream, whereas an ointment will generally contain a substantially higher proportion of oil phase compared to water phase, for example greater than 60 wt. % oil phase, preferably greater than 70 wt. % oil phase, more preferably greater than 80 wt. % oil phase, based on the total weight of the oil phase and the water phase. A lotion will generally contain a lower proportion of oil phase than a cream, for example under 25 wt. % oil phase, under 20 wt. % oil phase, under 15 wt. % oil phase, under 10 wt. % oil phase or under 5 wt. % oil phase, based on the total weight of the oil phase and the water phase.

Generally, a cream for use according to the invention comprises an oil phase and a water phase mixed together to form an emulsion. Preferably, the amount of water present in a cream of the invention is about 45% to about 85% by weight based on the total weight of the cream, more preferably about 45 wt. % to about 65 wt. %, even more preferably about 45 wt. % to about 55 wt. %.

Preferably, an emulsion for use according to the invention will be stabilised by a suitable, physiologically-acceptable surfactant to reduce the physical instability of the emulsion.

Where the composition is an ointment a physiologically-acceptable ointment base will be used. Examples of ointment bases include hydrocarbon bases such as such as hard paraffin, soft paraffin, ceresine and microcrystalline wax, absorption bases such as lanolin and beeswax, water-soluble bases such as polyethylene glycols (e.g. polyethylene glycol 200, 300, 400, 3350, 4000 or 6000), propylene glycol and polypropylene glycols, emulsifying bases such as emulsifying wax and cetrimide, and vegetable oils such as olive oil, coconut oil, sesame oil, almond oil and peanut oil. Mixtures of ointment bases can of course be used. The amount of ointment base present in an ointment of the invention is preferably about 60% to about 95% by weight based on the total weight of ointment, more preferably about 70 wt. % to about 90 wt. %, still more preferably about 75 wt. % to about 85 wt. %.

The composition for use in accordance with the present invention may also be a lotion containing the active component suspended or dissolved in one or more physiologically acceptable carriers. Particular carriers include, for example, mineral oil, sorbitan monostearate, polysorbate 60, cetyl esters wax, cetearyl alcohol, benzyl alcohol, 2-octyldodecanol and water.

Parenteral administration to patients can be by various routes including, but not limited to, subcutaneous, intravenous (including bolus injection), intramuscular, and intraarterial. Because their administration typically bypasses patients' natural defences against contaminants, compositions for parenteral administration are preferably sterile or capable of being sterilized prior to administration to a patient. Examples of such compositions include, but are not limited to, solutions ready for injection, dry products ready to be dissolved or suspended in a physiologically acceptable vehicle for injection, suspensions ready for injection, and emulsions.

Some suitable vehicles that can be used to provide compositions for parenteral administration include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Suitable dosages of active ingredient may be determined by a skilled medical practitioner. Actual dosage levels of active ingredient may be varied so as to obtain an amount of the active ingredient which is effective to achieve the desired therapeutic response for a particular patient, composition, and mode of administration, without being toxic to the patient. Thus, the dosage is typically an effective or therapeutically effective dosage.

The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response. For example, a single dose may be administered (e.g. a single dose daily), several divided doses may be administered over time or the dose may be proportionally reduced or increased as indicated by the exigencies of the therapeutic situation. Unit dose form (or, equivalently, “dosage unit form”) as used herein refers to physically discrete units suited as unitary dosages for the individuals to be treated; each unit dose contains a predetermined quantity of active compound calculated to produce the desired therapeutic effect in association with the required carrier.

In the compositions and unit doses described herein, the active ingredients may each, for example, be present at a concentration of between 0.001 and 100% by weight, relative to the total weight of the composition or product, preferably between 0.01 and 80%, more preferably between 1 and 70% by weight, and more preferably still between 20 and 50% by weight.

In one currently preferred embodiment, the 2,3-butanediol or ester, acetal or ketal thereof is to be administered to the individual at a daily dose in the range 10 to 2000 mg/kg, more preferably 50 to 1000 mg/kg and more preferably still 100 to 500 mg/kg, such as about 250 mg/kg (preferably all expressed with respect to mass of the 2,3-butanediol part of the active agent in the case of a physiologically acceptable ester, acetal or ketal thereof). The 2,3-butanediol or ester, acetal or ketal thereof may, for example, be administered over a period of at least one week, preferably at least two weeks, more preferably at least three weeks (e.g. at dosing amounts such that the mean daily dose corresponds to the amounts defined above). There is no upper limit on the period over which dosing may occur, although in some embodiments the 2,3-butanediol or ester, acetal or ketal thereof may be administered for a period of up to one year, for example up to six months, such as up to three months. Periods in which dosing is paused (“breaks”), e.g. before recommencing dosing, are also envisaged. In preventative treatments (i.e. when the active agent is to be used for prevention of a particular condition), dosing can in general occur for an indefinite period or until such time as a medical professional determines that the risk of development of a particular condition has receded. When the active agent is to be used for treatment, e.g. curative treatment, of a particular condition then the dosing can in general occur until the symptoms giving rise to diagnosis of the particular condition have receded or disappeared, e.g. as assessed by a medical professional.

The present invention is explained in more detail in the following by referring to the Examples, which are not to be construed as limitative.

### EXAMPLES

### Example 1

### Background

Previous studies (described in detail in Scott et al., Drug Metab Dispos 45:982-989 2017, the contents of which are hereby incorporated by reference in their entirety) have demonstrated that the flavin-containing monooxygenase FMO5 is a regulator of body weight and of glucose disposal and insulin sensitivity in mice. In particular, the study demonstrated that homozygous knockout (Fmo5^{-/-}) mice were, in comparison with wild-type (WT) counterparts, resistant to age-related changes in glucose homeostasis and maintain the higher glucose tolerance and insulin sensitivity characteristic of young animals. When fed a high-fat diet, they were protected against weight gain and reduction of insulin sensitivity. Fmo5^{-/-} mice were found to have metabolic characteristics similar to those of germ-free mice, indicating that FMO5 plays a role in sensing or responding to gut bacteria. In WT mice, FMO5 is present in the mucosal epithelium of the gastrointestinal tract where it is induced in response to a high-fat diet. In comparison with WT mice, Fmo5^{-/-} mice were found to have fewer colonic goblet cells, and to differ in the production of the colonic hormone resistin-like molecule β. Fmo5^{-/-} mice were found to have lower concentrations of tumor necrosis factor α in plasma and of complement component 3 in epididymal white adipose tissue, indicative of improved inflammatory tone. These results implicated FMO5 as a regulator of body weight and of glucose disposal and insulin sensitivity.

The knockout of FMO5 was thus found to give a remarkable phenotype characterised by: decreased body mass with age; lower fat mass with age; reduced plasma cholesterol; increased insulin sensitivity; not physically more active; eating more with age; and decreased metabolic ageing. The knockout mice were otherwise normal and long-lived.

The experiments detailed below relate to work done to establish a drug candidate that replicates, in wild-type mice, the benefits conferred by FMO5 gene knock-out.

### Discovery of 2,3-Butanediol as a Biomarker in the Fmo5^{-/-} Mice Model

Untargeted metabonomics was used to compare the metabolite profiles of the urine and plasma from Fmo5^{-/-} mice with those of age-matched WT mice at 8, 15, 30, 45 and 60 weeks of age.

600 MHz ¹H NMR spectroscopy was used for metabolite detection, quantification and identification. Visual inspection and multivariate statistical analysis showed that at all ages studied, the most distinctive difference between the profiles of the urines of age-matched Fmo5^{-/-} and WT mice was the presence of complex signals at ca 1.15 ppm in the ¹H NMR spectra of the Fmo5^{-/-} mice: these signals were almost always below the level of detection in the spectra of the urines of the corresponding WT mice.

A variety of two-dimensional NMR experiments were used to more fully analyse the origins of these signals, including 2D ¹H J-Resolved NMR Spectroscopy (JRES), 2D ¹H chemical shift correlated spectroscopy (COSY), 2D ¹³C, ¹H Heteronuclear Single Quantum Correlation (HSQC) and 2D ¹³C, ¹H Heteronuclear Multiple Bond Correlation (HMBC). These experiments conclusively demonstrated that the complex signals at ca 1.15 ppm were in fact overlapping, second-order, methyl signals at 1.146 and 1.149 ppm from the meso- and enantiomeric-isomers respectively of 2,3-butanediol, and typically in a ratio of ca 3:1 respectively. It was not determined whether the enantiomeric-2,3-butanediol was the 2S, 3S- or the 2R, 3R-isomer, or a mixture of these enantiomeric forms. However, since the 2,3-butanediol was shown to be largely originating from the mouse gut microbiome and since most bacteria produce 2R, 3R-butanediol, it appears likely that the enantiomeric isomer is 2R, 3R-butanediol (HMDB0033007, (2R,3R)-2,3-butanediol, InChI=1S/C4H10O2/c1-3(5)4(2)6/h3-6H,1-2H3/t3-,4-/m1/s1) and not 2S, 3S-butanediol ((2S,3S)-2,3-butanediol, InChI=1S/C4H10O2/c1-3(5)4(2)6/h3-6H,1-2H3/t3-,4-/m0/s1). The meso-isomer of 2,3-butanediol is also known as (2R,3S)-2,3-butanediol, InChI=1S/C4H10O2/c1-3(5)4(2)6/h3-6H,1-2H3/t3-,4+.

These NMR spectral assignments were confirmed by spiking authentic samples of meso-2,3-butanediol and 2R, 3R-butanediol into a sample of urine from a week 30 Fmo5^{-/-} mouse and rerunning the 600 MHz ¹H NMR spectra. These spiked spectra showed complete coincidence of all of the signals of the metabolites, including the second-order spectral features. Finally, high-resolution, ultra-performance liquid chromatography-time-of-flight mass spectrometry (HR UPLC-TOF-MS) analysis of the week 30 urines from both Fmo5^{-/-} and WT mice provided unequivocal and orthogonal confirmation of the NMR-based identification of these metabolites, based on matching UPLC retention times and mass spectral features.

Table 1 shows the key NMR data for the authentic reference standards of 2,3-butanediol and those data observed in Fmo5^{-/-} mouse urine

TABLE 1
NMR Data for 2,3-butanediol standards in
buffer and for mouse biomarkers in urine

| Sample | δ_{H} ¹ | δ_{C} ² | COSY | HMBC³ |
|---|---|---|---|---|
| authentic meso-23BD CH₃ | 1.143 | 19.3 | 3.738 | |
| authentic meso-23BD CH | 3.738 | 73.7 | 1.143 | |
| authentic 2R,3R-23BD CH₃ | 1.147 | 20.5 | 3.627 | |
| authentic 2R,3R-23BD | 3.627 | 74.2 | 1.147 | |
| meso-23BD CH₃ in week 30 | 1.146 | 19.6 | 3.73 | 73.6 |
| FMO5 KO mouse | | | | |
| enantiomeric-23BD CH₃ in | 1.149 | 20.7 | 3.63 | 74.5 |
| week 30 FMO5 KO mouse | | | | |

Footnotes:
¹ all proton signals in both isomers of 2,3-butanediol are 2^{nd} order multiplets.
² δ_{C} be values from HSQC spectra.
³HMBC indicates long range ¹³C to ¹H connectivity found in HMBC spectra of mouse urine.
4. all reference standard data for samples in pH 7.4 deuterated phosphate buffer.

As expected, 2,-3-butanediol was also detected by 600 MHz ¹H NMR spectroscopy in the plasma of the FMO5 KO mice, and it was undetectable in the corresponding plasma of age-matched WT mice. However, the levels in the FMO5 KO plasma were low and although the signals were discriminating between FMO5 KO and WT mouse plasma with a simple p=0.05 cut-off, they were broad, difficult to quantitate and difficult to assign to any particular isomer.

The other metabolite that exhibited statistically significantly different urinary levels across all time points measured except week 15, was taurine, although this metabolite was present in the urines of both FMO5 KO and WT mice. This metabolite was confidently identified by 1D ¹H NMR chemical shifts, coupling constants and multiplicities, 2D ¹H COSY, 2D ¹³C, ¹H HSQC and 2D ¹³C, ¹H HMBC NMR data and comparison of those data with that present in the human metabolite database (HMDB) for HMDB 00251 (taurine or 2-aminoethane-1-sulfonic acid, InChI=1S/C2H7NO3S/c3-1-2-7(4,5)6/h1-3H2,(H,4,5,6)). At weeks 30, 30 (set 2, duplicate group for validation), 45 and 60, but not at week 15, the levels of urinary taurine in FMO5 KO mice were lower than those in WT mice. In addition, statistical correlation spectroscopy (STOCSY) demonstrated that across all of the FMO5 KO urine samples, that the levels of taurine were significantly and negatively correlated with those of 2, 3-butanediol. STOCSY is a methodology that can determine which metabolites in a complex biological fluid such as urine have concentrations that are correlated with one another (see Statistical Spectroscopic Tools for Biomarker Discovery and Systems Medicine by Robinette, Lindon and Nicholson, Anal. Chem. (2013), 85, 5297-5303, dx.doi.org/10.1021/ac4007254).

It was hypothesised that the lower levels of urinary taurine excreted in the FMO5 KO mice indicate higher systemic levels of this protective metabolite. In support of this hypothesis, preliminary desorption electrospray ionisation (DESI) mass spectrometric imaging demonstrated higher levels of taurine and lower levels of hypotaurine, the precursor of taurine, in the liver as well as the colon of week 30, FMO5 KO mice relative to their WT counterparts. The absence of statistically significantly different urinary taurine levels in the FMO5 KO mice at week 15 is consistent with the clinical phenotype of the FMO5 KO mice, which does not becomes fully apparent until week 30.

Taurine has been shown to have a number of beneficial effects including reducing weight gain, reducing fat, increasing insulin sensitivity and reducing cholesterol (Murakami, S.; Kondo, Y.; Nagate, T., Effects of long-term treatment with taurine in mice fed a high-fat diet—Improvement in cholesterol metabolism and vascular lipid accumulation by taurine. In Taurine 4: Taurine and Excitable Tissues, DellaCorte, L.; Huxtable, R. J.; Sgaragli, G.; Tipton, K. F., Eds. 2000; Vol. 483, pp 177-186.; Murakami, S., Taurine and atherosclerosis. Amino Acids 2014, 46 (1), 73-80.; Murakami, S.; Fujita, M.; Nakamura, M.; Sakono, M.; Nishizono, S.; Sato, M.; Imaizumi, K.; Fukuda, N., Mechanism of the cholesterol-lowering effect of taurine. Amino Acids 2015, 47 (8), 1653-1654.; Murakami, S., Role of taurine in the pathogenesis of obesity. Molecular Nutrition & Food Research 2015, 59 (7), 1353-1363.; Murakami, S.; Fujita, M.; Nakamura, M.; Sakono, M.; Nishizono, S.; Sato, M.; Imaizumi, K.; Mori, M.; Fukuda, N., Taurine ameliorates cholesterol metabolism by stimulating bile acid production in high-cholesterol-fed rats. Clinical and Experimental Pharmacology and Physiology 2016, 43 (3), 372-378.; Sagara, M.; Murakami, S.; Mizushima, S.; Liu, L. J.; Mori, M.; Ikeda, K.; Nara, Y.; Yamori, Y., Taurine in 24-h Urine Samples Is Inversely Related to Cardiovascular Risks of Middle Aged Subjects in 50 Populations of the World. In Taurine 9, Marcinkiewicz, J.; Schaffer, S. W., Eds. 2015; Vol. 803, pp 623-636.; Imae, M.; Asano, T.; Murakami, S., Potential role of taurine in the prevention of diabetes and metabolic syndrome. Amino Acids 2014, 46 (1), 81-88). All of these features are part of the phenotype of the FMO5 KO mouse and thus elevated systemic levels of taurine in the FMO5 KO mice may also contribute in part to the phenotype of the FMO5 KO mice.

In conclusion, the metabonomic analysis identified that the FMO5 knock-out mice excreted a substantial quantity of 2,3-butanediol in their urine, in the range of ca 10 to 100 ug per ml by quantitative NMR spectroscopy relative to an internal standard of 3-(trimethylsilyl)-2,2′,3,3′-tetradeuteropropionic acid (TSP). 2,3-butanediol was below the limits of the NMR detection used in wild-type control mice. 2,3-butanediol is quite readily detectable by NMR spectroscopy due to its low molecular weight and the presence of 6 chemically equivalent methyl protons, even though the methyl signal is second order due to molecular symmetry effects.

Fmo5^{-/-} mice were then dosed with two antibiotics in their drinking water: sodium ampicillin (AppliChem, 1 g/l) and neomycin (Sigma Aldrich, 0.5 g/1) and urine from the antibiotic-treated mice was collected on the 14^{th} day of treatment using standard methods.

The ¹H NMR spectra of the antibiotic-dosed Fmo5^{-/-} mice exhibited an ablation of microbiome-derived metabolites including hippurate, 3-indoxyl sulphate, cinnamoylglycine, 4-cresolsulphate, 4-cresolglucuronide and exhibited significantly-reduced levels (5 to 7-fold) of both isomers of 2,3-butanediol. It was thus concluded that the 2,3-butanediol was mainly microbiome-derived, i.e. produced by bacteria in the gut.

In the Fmo5^{-/-} mice treated with antibiotics, not only were the levels of the 23BD isomers reduced by 5- to 7-fold, but the low levels of plasma cholesterol in the Fmo5^{-/-} mice were found to have increased. This result indicated that 2,3-butanediol was both diagnostic of, and causative of, at least part of, the FMO5 KO phenotype. In order to prove that hypothesis, a series of experiments were performed dosing 23BD to WT mice to determine the effects of dosing on the phenotype of the WT mice (see below).

The results clearly showed the reduction in total cholesterol in the FMO5 knock-out mice and the reversal of that effect by antibiotic treatment, i.e. indicating the role of gut bacteria in the effects arising in the FMO5 knock-out mice.

### Efficacy of 2,3-Butanediol in WT Mice: Short Term Dosing Protocol

A preliminary dose-ranging experiment was first performed to generate concentrations of 2,3-butanediol in the urine of wild-type mice equivalent to those seen in the urine of the FMO5 knock-out mice. 8-week old male mice were dosed with 0, 60, 250 or 600 mg/kg of 2,3-butanediol in drinking water, n=4 per group.

Urine was collected at day 0 (pre-treatment) and at 4 days of treatment.

2,3-butanediol presence was assessed by NMR analysis of urine.

Analysis of variance (ANOVA) for the logarithm of the NMR-detected levels of urinary 23BD (measured on the methyl resonance at 1.150 ppm) in the 8 cohorts of C₅₇BL/6 mice studied: control day 0, 60 mg/kg day 0, 250 mg/kg day 0 and 600 mg/kg day 0 and then control day 28, 60 mg/kg day 28, 250 mg/kg day 28 and 600 mg/kg at day 28 of dosing with 2,3-butanediol in the drinking water. Day 0 is prior to commencement of dosing with 2,3-butanediol on day 1 and no 2,3-butanediol was detected.

2,3-butanediol was then administered over a four-week dosing period to wild-type mice. The 2,3-butanediol was delivered to male, wild-type C57BL/6 mice in drinking water, from 13 to 17 weeks of age. Dose delivery estimation was based on mouse average water consumption. Groups of male mice were either untreated, treated with 60, 250 or 600 mg/kg 2,3-butanediol in their drinking water. Water was changed twice weekly. Weights of mice and food intake were recorded. Urine was collected on day 0 (age 13 weeks, pre-treatment) and at 28 days post-treatment (mice aged 17 weeks) and analysed for 2,3-butanediol.

Blood was collected on day 0 (age 13 weeks) and at 28 days post-treatment (mice aged 17 weeks). Plasma was isolated and analysed. At termination of experiment, epidydimal fat pads (EWAT) were weighed and expressed as EWAT:Body weight ratios,

Epididymal white adipose tissue (EWAT) to body weight ratios were significantly reduced at a 250 mg/kg/day dose. 2,3-butanediol thus reduced fat depots.

Plasma cholesterol was significantly reduced at a 250 mg/kg/day dose.

Plasma triglycerides were significantly reduced at 250 and 600 mg/kg/day doses.

Plasma free fatty acids were significantly reduced at 250 and 600 mg/kg/day doses.

Plasma lactate dehydrogenase was significantly reduced at 250 and 600 mg/kg/day doses.

No changes were observed in any of plasma alkaline phosphatase, plasma alanine aminotransferase and plasma aspartate aminotransferase

STOCSY analysis for 23BD (using the resonance at 1.150 ppm as the ‘driver’ signal): The upper section of the STOCSY plot demonstrates metabolites with positive correlations to the peak at ca 1.150, and the lower part shows metabolites with negative correlations. This STOCSY NMR showed that the levels of 2,3-butanediol in the urines of the 23BD-treated WT mice were inversely correlated with those of taurine in the urine of these same mice. The same inverse correlation was observed in the urine of the FMO5 KO mice. The significance of this observation is that the treatment of WT mice with 23BD resulted in the same inverse correlation between

23BD and taurine concentrations that was observed in the Fmo5^{-/-} mice and thus the 2,3-butanediol treatment in the wildtype C57BL/6 mice is ‘phenocopying’, or creating a similar phenotype to that observed in the FMO5 KO mice.

### Efficacy of 2,3-Butanediol in WT Mice: Longer Term Dosing Protocol

Male mice were dosed with 200 mg/kg/day 2,3-butanediol from age 8 weeks to age 29 weeks. Mice were housed 4 to a cage. At age 22 weeks, treatment was removed from one cohort, called the ‘washout 6 weeks’. Treatment continued in one cohort, called ‘treatment throughout’. Urine collected 1 week following removal of 2,3-butanediol confirmed the absence of 2,3-butanediol in the washout cohorts. At the end of the experiment blood was collected and plasma isolated and analysed. Mice were culled, and final body weights and epidydimal fat deposits recorded.

The results indicated that 2,3-butanediol causes significant reductions in fat (EWAT/body weight) and plasma cholesterol. Furthermore, 2,3-butanediol appears to reprogramme fat and cholesterol biosynthesis, as the treatment effects were found to be persistent through a 6 week washout, when the mice were being treated with water alone and no 2,3-butanediol.

### CONCLUSIONS

Metabonomic analysis identified a microbiome-derived compound, 2,3-butanediol, at high concentrations specifically in the urine and plasma of FMO5 knock-out mice. Treatment of wild-type mice with 2,3-butanediol ‘phenocopied’ several features of the FMO5 knock-out mice. For example, 2,3-butanediol treatment of these wildtype (C57BL/6) mice caused significant reductions in: fat to body weight ratio; plasma cholesterol triglycerides; free fatty acids and lactate dehydrogenase. In addition, as was found in the Fmo5^{-/-} mice, taurine levels were inversely correlated with the levels of 2,3-butanediol and were reduced relative to untreated wildtype mice. These results indicate that 2,3-butanediol may be efficacious in treatment of conditions such as obesity, insulin resistance and diabetes.

Without being limited to theory, the mode of action may involve inhibition of glucose uptake from the gastrointestinal (GI) tract via an effect on glucose transporters. Evidence in support of this hypothesis includes the following.
1. Literature evidence, which includes: (i) Lomeo et al (Diabetes, 37, 912-915 (1988)), where it was demonstrated that 2,3-butanediol was a potent inhibitor of ¹⁴C-glucose incorporation into triglyceride fatty acid and, at higher concentrations, triglyceride glycerol into Sprague-Dawley rat adipocytes; (ii) Paoli et al (Eur J Clin Nutrition (2013)), where it is stated that, under conditions of sustained glucose deprivation, ketone bodies “begin to be utilized as an energy source by the CNS when they reach a concentration of about 4 mmol/l, which is close to the Km for the monocarboxylate transporter” (thus, it is possible that 2,3-butanediol, like the ketone bodies mentioned, has an effect on glucose transport from the GI tract via an affinity to its transporter.
2. 2,3-butanediol-glucose transporter interactions may inhibit glucose uptake from the gut to the host and divert glucose utilization to the microbiome and/or to waste via excretion in the faeces.
3. The partial, potential pharmacophore overlap between one conformation (others are available via single bond rotation) of 2R, 3R-butanediol and D-glucose (see depiction of beta-D-glucose below) is also striking—see below—which hints at the possibility that this the moiety may be responsible for interacting with glucose transporters (also note that, whilst this analysis compares 2,3-butanediol with C3 and C4 of glucose, the same argument would apply in a comparison with C2 and C3).

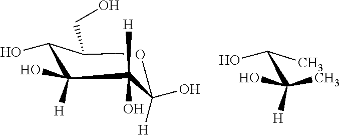

4. This idea also has support from plant metabolic profiling studies (Effantin et al Molecular Microbiology (2011): doi:10.1111/j.1365-2958.2011.07881.x) where it is stated that infection by pathogens such as bacteria of the genera Dickeya and Pectobacterium give rise to “a decline in plant sugars and amino acids during infection and the concomitant appearance of a compound identified as 2,3-butanediol”, this being suggestive of a parallel between plants and mammals.
5. It is also possible that other mechanisms are operating in parallel: for example, earlier work shows increased utilization of glucose as fuel, relative to fat, in FMO5 KO mice (Gonzalez Malagon et al. 2015, Biochem Pharmacol http://dx.doi.org/10.1016/j.bcp.2015.05.013). However, the proteomics results show decreases in three enzymes involved in carbohydrate metabolism: ALDOB, GPD1 and cytosolic ME1, which could be caused by lowering of glucose availability. (Gonzalez Malagon et al. 2015, Biochem Pharmacol http://dx.doi.org/10.1016/j.bcp.2015.05.013). Cytosolic ME1 provides NADPH for the synthesis of cholesterol and fatty acids.

Humans and mice have orthologous glucose transporters. Additionally, it is already known that FMO5 can be repressed in type 2 diabetics in humans (Takamura et al 2004, Diabetologia 47:638-647 doi 10.1007/s00125-004-1366-y) and that the expression patterns of FMO5 in humans and mice are very similar (in both species the protein is present in the liver and in the gut). These considerations lend confidence to the transferability of the results in mice described herein to human subjects.

1.-12. (canceled)

13. A unit dose form suitable for oral administration to an individual, wherein the unit dose form contains 2,3-butanediol, or a pharmaceutically acceptable salt of 2,3-butanediol, or a physiologically acceptable ester, acetal or ketal of 2,3-butanediol, in an amount of from 1 to 50 g, together with at least one physiologically acceptable excipient or carrier.

14. The unit dose form of claim 13, wherein the individual is a human, cat, dog or horse.

15. The unit dose form of claim 13, wherein the unit dose form is formulated as a liquid.

16. The unit dose form of claim 13, wherein the at least one physiologically acceptable excipient or carrier comprises at least one of a flavouring agent, a viscosity modifier, a surfactant, a preservative and a colouring agent.

17. A method of treating or preventing a condition selected from the group consisting of obesity, insulin resistance and diabetes in an individual in need thereof, the method comprising administering to the individual a therapeutically effective amount of 2,3-butanediol, or a pharmaceutically acceptable salt of 2,3-butanediol, or a physiologically acceptable ester, acetal or ketal of 2,3-butanediol.

18. A method of controlling the weight of an individual or reducing the metabolic ageing of an individual, the method comprising administering to the individual 2,3-butanediol, or a pharmaceutically acceptable salt of 2,3-butanediol, or a physiologically acceptable ester, acetal or ketal of 2,3-butanediol, in an amount sufficient to control said weight.

19. (canceled)

20. The method of claim 17, wherein the individual is a human, cat, dog or horse.

21. (canceled)

22. The method of claim 18, wherein the individual is a human, cat, dog or horse.
